(19) United States
(12) Patent Application Publication
(10) Pub. No.: US 2014/0316255 A1
(43) Pub. Date: Oct. 23, 2014

(54) Raman Imaging Devices and Methods of Molecular Imaging

(71) Applicants: Ellis Garai, Sherman Oak, CA (US); Cristina Zavaleta, Palo Alto, CA (US); Michael Mandella, Palo Alto, CA (US); Jonathan Liu, Port Jefferson, NY (US); Sanjiv Sam Gambhir, Portola Valley, CA (US); Christopher H. Contag, San Jose, CA (US)

(72) Inventors: Ellis Garai, Sherman Oak, CA (US); Cristina Zavaleta, Palo Alto, CA (US); Michael Mandella, Palo Alto, CA (US); Jonathan Liu, Port Jefferson, NY (US); Sanjiv Sam Gambhir, Portola Valley, CA (US); Christopher H. Contag, San Jose, CA (US)

(21) Appl. No.: 14/241,790

(22) PCT Filed: Aug. 23, 2012
(86) PCT No.: PCT/US2012/051999

§ 371 Date: Jun. 10, 2014

Related U.S. Application Data
(60) Provisional application No. 61/530,598, filed on Sep. 2, 2011.

(51) Int. Cl. A61B 5/00; A61B 1/07; A61B 10/02; A61K 49/00; A61B 19/00

(52) U.S. Cl. CPC A61B 5/0075; A61K 49/0095; A61B 5/0079; A61B 5/0084; A61B 19/5244; A61B 10/02; A61B 1/07; A61B 2019/5257
USPC 600/424; 600/431; 600/478

(57) ABSTRACT

In accordance with the purpose(s) of the present disclosure, as embodied and broadly described herein, embodiments of the present disclosure, in one aspect, relate to Raman imaging devices (e.g., Raman endoscope probes) or systems, methods of using Raman agents, Raman imaging devices, and/or systems to image or detect a signal, and the like.

### CROSS-REFERENCE TO RELATED APPLICATION

This application claims priority to U.S. provisional application entitled “RAMAN IMAGING DEVICES AND METHODS OF MOLECULAR IMAGING,” having Ser. No. 61/530,598 filed on Aug. 31, 2011, which is entirely incorporated herein by reference.

### BACKGROUND

Early detection remains one of the most powerful ways to improve prognosis for cancer patients. As a result, a significant effort has been made to develop new diagnostic strategies to detect early stage cancer more sensitively, both in-vitro and in-vivo. Raman spectroscopy has proven to be a powerful analytical tool that offers unsurpassed sensitivity and multiplexing capabilities. Harnessing these unique properties for early detection of cancer could serve as a powerful diagnostic strategy, with the potential to significantly impact the survival rate of those patients diagnosed earlier. However, current approaches are problematic and need to be overcome.

### SUMMARY

Embodiments of the present disclosure, in one aspect, relate to Raman imaging devices (e.g., Raman endoscope probes) or systems, methods of using Raman agents, Raman imaging devices, and/or systems to image or detect a signal, and the like.

An embodiment of the method of imaging, among others, includes: administering at least a first type of Raman agent to a subject, wherein the Raman agent has an affinity for a specific target; introducing a Raman imaging device to an area of the subject; exposing the area to a light beam from the Raman imaging device, wherein the light beam is scattered by the first type of Raman agent that is associated with the specific target, wherein the light beam that is scattered is referred to as a Raman scattered light energy; detecting the Raman scattered light using the Raman imaging device; and using the Raman scattered light energy to form an image.

An embodiment of the method of performing Raman imaging, among others, includes: providing, simultaneously, an untargeted Raman agent and a targeted Raman agent to a subject; and evaluating the ratio of Raman scattered light signals from the targeted and the untargeted Raman agents in an area, wherein the ratio provides an estimated measurement of truly bound Raman agents, wherein the measurement is substantially independent of the free-space optical working distance to the sample.

An embodiment of the method of imaging, among others, includes: introducing a Raman imaging device to the subject; positioning the Raman imaging device adjacent the specific target; exposing the area to a light beam from the Raman imaging device, wherein the light beam is scattered by the tissue in the area, wherein the light beam that is scattered is referred to as Raman scattered light energy; and detecting the Raman scattered light using the Raman imaging device, using the Raman scattered light energy to form an image.

An embodiment of the Raman imaging system for inspection of a sample, among others, includes: a light source; a Raman detection system; an optical fiber system to guide light derived from the light source to the sample and to further guide Raman scattered light energy from the sample to the Raman detection system; and an optic system between the optical fiber system and the sample to concentrate said light onto the sample and to further collect the Raman scattered light energy from the sample, wherein the optic further concentrates the collected Raman scattered light energy onto the optical fiber system.

An embodiment of the Raman imaging system for inspection of a sample, among others, includes: a light source; a Raman detection system; a first optical fiber system to guide light from the light source to the sample; a second optical fiber system to guide Raman scattered light energy from the sample to the Raman detection system, wherein, the second optical fiber system has a proximal end adjacent to said Raman detection system; and a first optic to concentrate said light onto the sample along a first axis.

### BRIEF DESCRIPTION OF THE DRAWINGS

Many aspects of the disclosed compositions and methods can be better understood with reference to the following drawings. The components in the drawings are not necessarily to scale, emphasis instead being placed upon clearly illustrating the relevant principles. Moreover, in the drawings, like reference numerals designate corresponding parts throughout the several views.

FIG. 1 illustrates that the SERS nanoparticles can be coated with one or more types of tumor targeting agent.

FIGS. 2A and 2B illustrate a method of using an embodiment of the present disclosure.

FIG. 3 illustrates an embodiment of a Raman endoscope designed to be inserted through the accessory channel of a clinical endoscope.

FIG. 4 illustrates an embodiment of the system.

FIG. 5 illustrates the final fabricated Raman endoscope to be used for clinical studies.

FIG. 6A shows an embodiment of the present disclosure, while FIG. 6B illustrates an embodiment of an optical breadboard from the embodiment shown in FIG. 6A.

FIG. 7 illustrates a representation of a received signal and its graph representation of the signal.

FIG. 8 illustrates the analysis of a signal.

FIG. 9A illustrates a graph depicting stable power output over a working distance of 25 mm away from sample surface.

FIG. 9B illustrates a graph showing excellent Raman signal reproducibility of our Raman endoscope over several integration times.

FIG. 9C illustrates a graph showing stable Raman signal over a working distance of 10 mm away from sample surface

FIG. 9D is a graph showing that our Raman endoscope is able to detect Raman signal at depths of 4-5 mm when using I sec integration times in our tissue mimicking phantom (photo at right).

FIG. 9E is a graph showing the sensitivity of our Raman endoscope where the limit of detection was 326 fM (15 mW at 1 s) and 440 fM (42 mW at 300 ms) of SERS nanoparticles in a well plate (photo at right).

FIG. 9F is a graph depicting the sensitivity of our Raman endoscope after topically applying diluted concentration of SERS nanoparticles onto fresh human colon tissue samples (photo at right).

FIG. 10A illustrates ten unique types (flavors) of SERS nanoparticles spatially separated onto a piece of quartz.

FIG. 10B illustrates an equal mixture of S440 and one other flavor is placed in separate drops across a piece of quartz to characterize dual colocalization of SERS nanoparticles.

FIG. 10C illustrates a demonstration of multiple colocalized SERS flavors including mixtures of 4, 6, 8 and all 10 SERS nanoparticles within the same droplet on quartz.

FIG. 10D illustrates a mix of 4 SERS nanoparticle flavors each at varying concentrations.

FIG. 11A illustrates ten unique flavors of SERS nanoparticles spatially separated onto 10 separate pieces of fresh human colon tissue.

FIG. 11B illustrates a demonstration of colocalized multiplexing, where 4 SERS flavors were equally mixed and applied on a single piece of human colon tissue.

FIG. 11C illustrates a mix of 4 SERS nanoparticle flavors each at varying concentrations were mixed together and applied to a single piece of human colon tissue.

FIG. 12 illustrates that the Raman signal can be evaluated over a variety of working distances and for a variety of power and integration times.

FIG. 13 illustrates a graph of working distance vs. Raman signal.

FIG. 14 illustrates a graph depictings a linear trend where Raman signal increases linearly with increased laser power settings.

FIG. 15A illustrates an embodiment of a Raman endoscope inserted into the instrument channel of a conventional clinical colonoscope.

FIG. 15B illustrates a magnified digital photograph taken from the white light endoscopy component of the clinical colonoscope portraying our Raman endoscope protruding from the instrument channel and illuminating a spot on the colon wall in a human patient.

FIGS. 16A and 16B illustrate images that show the effect of using a calibrated ratio of the signals approach for the quantification of biomarker expression and the effective suppression of nonspecific signals and background.

FIG. 17 illustrates a schematic of an embodiment of the present disclosure that uses a 45-deg scanning mirror to perform circumferential scans of the lumen of the colon.

FIG. 18 illustrates another embodiment of the present disclosure.

FIGS. 19 and 20 illustrate that embodiments of the present disclosure can be used to distinguish a variety of tissues of interest in the colon such as: tubular adenomas, villous adenomas, tubulovillis adenomas, flat lesions, mucosal carcinoma in-situ, submucosal carcinoma in-situ, and more advanced carcinomas.

FIG. 21 illustrates a schematic of an embodiment of the present disclosure.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biochemistry, biology, molecular biology, imaging, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and I atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise. Thus, for example, reference to “a compound” includes a plurality of compounds. In this specification and in the claims that follow, reference will be made to a number of terms that shall be defined to have the following meanings unless a contrary intention is apparent.

### Definitions

In describing and claiming the disclosed subject matter, the following terminology will be used in accordance with the definitions set forth below.

The term “Surface-Enhanced Raman Scattering (SERS)” refers to the increase in Raman scattering exhibited by certain molecules in proximity to certain metal surfaces. (see, U.S. Pat. No. 5,567,628) The SERS effect can be enhanced through combination with the resonance Raman effect. The surface-enhanced Raman scattering effect is even more intense if the frequency of the excitation light is in resonance with a major absorption band of the molecule being illuminated. In short, a significant increase in the intensity of Raman light scattering can be observed when molecules are brought into close proximity to (but not necessarily in contact with) certain metal surfaces. In an embodiment, the metal surfaces can be “roughened” or coated with minute metal particles. Metal colloids also show this signal enhancement effect. The increase in intensity can be on the order of several million-fold or more.

The term “reporter compound” can refer to a Raman-active label. The term “Raman-active label” can refer to a substance that produces a detectable Raman spectrum, which is distinguishable from the Raman spectra of other components present, when illuminated with a radiation of the proper wavelength.

As used herein, the term “Raman agent” refers to the compounds or structures of the present disclosure that are capable of serving as imaging agents either alone or in combination with attached molecules (e.g., antibodies, proteins, peptides, small organic molecules, aptamers, and the like).

The term “administration” refers to introducing a Raman agent (or a compound, cell, or virus, including the Raman agent) of the present disclosure into a subject. The preferred route of administration of the compounds is intravenous. However, any route of administration, such as oral, topical, subcutaneous, peritoneal, intraarterial, inhalation, vaginal, rectal, nasal, introduction into the cerebrospinal fluid, or instillation into body compartments can be used. In an embodiment, the Raman agent is administered locally (e.g., colon) so that it is not systemically distributed throughout the body.

In accordance with the present disclosure, “a detectably effective amount” of the Raman agent (e.g., SERS nanoparticle) of the present disclosure is defined as an amount sufficient to yield an acceptable image using equipment that is available for pre-clinical or clinical use. In an embodiment, a detectably effective amount of the Raman agent of the present disclosure may be administered in more than one injection. The detectably effective amount of the Raman agent of the present disclosure can vary according to factors such as the degree of susceptibility of the individual, the age, sex, and weight of the individual, idiosyncratic responses of the individual, the dosimetry, and the like. Detectably effective amounts of the Raman agent of the present disclosure can also vary according to instrument and digital processing related factors. Optimization of such factors is well within the level of skill in the art.

As used herein, the term “subject” or “host” includes humans and mammals (e.g., mice, rats, pigs, cats, dogs, and horses,). Typical subjects to which compounds of the present disclosure may be administered will be mammals, particularly primates, especially humans. For veterinary applications, a wide variety of subjects will be suitable, e.g., livestock such as cattle, sheep, goats, cows, swine, and the like; poultry such as chickens, ducks, geese, turkeys, and the like; and domesticated animals particularly pets such as dogs and cats. For diagnostic or research applications, a wide variety of mammals will be suitable subjects, including rodents (e.g., mice, rats, hamsters), rabbits, primates, and swine such as inbred pigs and the like. The term “living subject” refers to host or organisms noted above that are alive. The term “living subject” refers to the entire host or organism and not just a part excised (e.g., a liver or other organ) from the living subject.

As used herein, the term “in vivo imaging” refers to imaging living subjects (e.g., human or mammals).

### Discussion

In accordance with the purpose(s) of the present disclosure, as embodied and broadly described herein, embodiments of the present disclosure, in one aspect, relate to Raman imaging devices (e.g., Raman endoscope probes) or systems, methods of using Raman agents, Raman imaging devices, and/or systems to image or detect a signal, and the like.

Embodiments of the present disclosure seek to improve detection of a disease or condition during conventional endoscopic, laparoscopic, intraoperative, or surgical procedures. Embodiments of the present disclosure can accomplish this through molecular imaging using a Raman imaging device. In an embodiment, the Raman imaging device can be a Raman endoscope probe that can be used in an endoscope, a handheld Raman spectroscopy imaging device, or a low profile, minimally invasive imaging system. The molecular imaging can be accomplished by using one or more types of Raman agents, where each produce a Raman light scattering signal, and a Raman imaging device, which can excite the Raman agents with light and sensitively detect Raman scattered light energy signals emitted from the Raman agent(s). Embodiments of the Raman imaging device can be used in conjunction with Raman agents that target a specific disease to detect it earlier and at its margins with greater sensitivity than what is currently used.

In an embodiment, the Raman agents that have an affinity for a target can be used with Raman agents that are non-specific (e.g., uncoated or coated with agents that are non-specific). This combination can be useful when it is difficult to uniformly dispose the Raman agents on the area of interest and/or when it is difficult to wash the unattached Raman agents form one or more portions of the area (e.g., an area that may include tissue folds such as in the colon). In an embodiment, a ratio of a first type of Raman agents that have an affinity for a target and a second type of Raman agent that is not specific for a target (untargeted Raman agent) can be used to determine the presence and location of a target.

In an embodiment, the molecular imaging can be accomplished by using non-Raman agents and/or the tissue itself (e.g., can inherently produced Raman light scattering). In an embodiment, the non-Raman agents or tissue can produce a Raman light scattering signal and the Raman imaging device can detect Raman scattered light energy signals.

Embodiments of the present disclosure include a diagnostic tool (e.g., Raman imaging device such as a Raman endoscope probe or handheld device) and methods for identification of a disease or condition in subjects (e.g., human) who are undergoing a surgical, laparoscopic, intraoperative, or endoscopic procedure, where a device including the Raman imaging device (such an endoscope including the Raman endoscope probe or handheld device) is inserted into the body (e.g., cervix, bladder, bronchioles, esophagus, stomach, colon, rectum, skin, oral mucosa, and intraoperatively or laparoscopically into an organ, and the like) or placed over the region of interest (during surgical or intraoperative procedures). Raman agents can be conjugated with one or more disease targeting ligands and administered to the subject. The targeted Raman agents then sensitively and specifically bind to the cells, proteins, and the like, related to the disease or condition of interest and their localization can be detected using the Raman imaging device. The technique acts as an in-vivo histopathological tool assisting the physician to immediately identify a diseased area and its margins without having to involve a third party pathologist.

The principle by which embodiments of the present disclosure operate is based on the Raman Effect. When light is scattered from a molecule most photons are elastically scattered. However, a small fraction of light is scattered at optical frequencies different from and usually lower than the frequency of the incident photons. The process leading to this inelastic scatter is termed the Raman Effect. However, this effect is very weak, only producing one inelastically scattered photon for every 10 million elastically scattered photons. Therefore surface enhanced Raman scattering (SERS) agents will be used. SERS is a plasmonic effect where small molecules adsorbed onto a nano-roughened noble metal surface, for example, experience a dramatic increase in the incident electromagnetic field resulting in several orders of magnitude higher Raman intensity. The increase in the Raman Effect allows embodiments of the present disclosure to detect pM concentrations of Raman agents with the Raman imaging device. The Raman agents can be selected so that they include unique Raman active molecules (that can be interchanged for multiplexing capabilities) adsorbed onto a metal core. In addition, the Raman agents can be conjugated to a disease targeting ligand that has an affinity for and a binding potential to the diseased area as opposed to normal tissue. Once the Raman agents have been conjugated to the appropriate disease targeting ligand, the Raman agents can be administered to the subject and the Raman agents are given an appropriate amount of time to bind to the targeted disease (e.g., diseased tissue or cells or compounds associated with the disease). Subsequently, using the Raman imaging device, a light beam can be directed onto the area of interest (e.g., which may include the suspected diseased area) to detect inelastic scattering (Raman scattering light energy) coming from disease targeted Raman agents (or the tissue or non-Raman agents).

As mentioned above, embodiments of the present disclosure include using Raman agents to locate and detect a signal from a diseased area of interest. In an embodiment, the Raman agents give a much more intense Raman signal than the intrinsic Raman scattering from the tissues themselves (e.g., about 10⁷ orders of magnitude greater) allowing the achievement of at least pM sensitivity.

Embodiments of the Raman imaging device can take the form of several instruments such as, but not limited to, an endoscope, a handheld Raman imaging device, or even a microscope. In general, the Raman imaging device includes a light source (e.g., a laser) or is adapted to direct a light source (e.g., uses a fiber to guide the light) that may be generated separately from the Raman imaging device, and a device or structure to receive or detect Raman scattered light energy (e.g., uses a fiber to collect light).

Optionally the Raman imaging device includes one or more lenses to guide the light and the scattered Raman light energy, one or more mirrors to direct the laser light or scattered Raman light energy, and/or one or more filters to select certain wavelengths of light and/or scattered Raman light energy. The resulting light can then be measured by a device (e.g., a spectrometer/CCD).

In an embodiment, the Raman imaging device or a system including the Raman imaging device can include collection and measurement devices or instruments to collect and measure the scattered Raman light energy.

In an embodiment, the area of interest can be mapped using the Raman imaging device by circumferentially scanning an area during a controlled retraction. In an embodiment, the Raman imaging device can include the ability to oscillate one or more mirrors back and forth at one or more angles to scan a large area around the entire circumference of the Raman imaging device (e.g., scan the entire surface of the colon as the Raman imaging device passes through). In an embodiment, circumferential scanning capabilities allow a larger area to be scanned at once. In an embodiment, circumferential scanning combined with a controlled retraction can allow for a mapping of a hollow region (such as the colon, esophagus, cervix, etc.).

In an embodiment, the device can be used as a contact probe or a non-contact probe. In addition, an angled mirror can be used to image an area at an angle to the probe, for instance at an angle greater than about 45 degrees (e.g., about 45 to 90 degrees), such that the area being imaged does not have to be adjacent to the probe, but rather in front (or even behind) the probe. In an embodiment, having the mirror oscillate back and forth through a given angle allows for a large area to be scanned without moving the probe.

In a particular embodiment, once a diseased area is located, the mirrors and/or lenses can be used to image the area during the biopsy. In particular, once a region of interest is detected, the rotating mirror can be held at a certain angle and used to help guide biopsies or resection of tissue in real-time.

In an embodiment, a collimating lens can be used. In an embodiment, the collimating lens placement allows for a consistent Raman signal to be produced over a variety of working distances. In an embodiment, the probe can use a having an illumination range of about 300 nm to 2000 nm.

In an embodiment, the device does not necessarily need to use a “Raman agent”, but can also utilize the intrinsic (or natural) Raman signal of the tissue itself or it can use contrast agents (such as fluorophores).

In an embodiment, enabling software can display (in order to inform the user/physician) the relative signal strength of each Raman active agent as well as the ratio signal strength (e.g., of specific Raman targeting agent to non-specific Raman agent).

In an embodiment, the Raman imaging device can be a Raman endoscope probe, where Raman endoscope probe can be used with an endoscope. A Raman endoscope probe, as discussed in detail below, is but one embodiment, and other embodiments of the present disclosure are not limited to Raman endoscope probes and portions of the discussion below describing the principles of operation and use can be applied to other Raman imaging devices such as, but not limited to, those described herein.

In an embodiment, the device is able to be used in conjunction with currently available endoscopes. In an embodiment, the device can be inserted into the working channel of an endoscope. In an embodiment, a plurality of multi-mode fibers (e.g., 36) can be used to increase the flexibility of the fiber bundle so that it can be inserted and used in a fully articulating endoscope, while still being able to collect as much of the scattered Raman light as possible.

In general, an endoscope includes one or more channels down the length of the endoscope. At least one channel can accept the Raman endoscope probe. The Raman endoscope probe can be inserted into the endoscope before or after the endoscope is introduced into the subject.

Embodiments of the Raman endoscope probe system can include a fiber bundle, one or more lenses for collimating a light beam (e.g., a laser at a wavelength that the Raman agents scatter the light) and for focusing the Raman scattered light energy, and optionally filters for delivering and collecting the appropriate light signals. Other components of the Raman endoscope probe system include a spectrometer and charge-coupled device (CCD) camera for collection and measurement of inelastically scattered light. The fiber bundle can be used to direct the light and collecting Raman scattered light energy.

The Raman agents can include Raman compounds and Raman nanoparticles. In an embodiment, the Raman compounds can include reporter compounds conjugated with one or more distinct targeting agents, both of which are described in more detail below. In an embodiment, the Raman nanoparticles include, but are not limited to, SERS nanoparticles, composite organic inorganic nanoparticles (COINS), Single walled nanotubes (SWNTs), methylene blue dye (other Raman active dyes), and the like. Each of the Raman nanoparticles can include targeting ligands (e.g., proteins) so that targeted areas (e.g., organs (e.g., colon), and the like) can be imaged.

In an embodiment, the SERS nanoparticle includes, but is not limited to, a core, a reporter compound, and an encapsulant material. The encapsulant material covers and protects the core and reporter compounds. The reporter compounds are attached to the core. The core can be made of materials such as, but not limited to, copper, silver, gold, and combinations thereof, as well as of other metals or metalloids. Different types of SERS nanoparticles can be selected, where each SERS nanoparticle has a different Raman signature. Thus, the use of different SERS nanoparticles enables multiplexing. Additional details regarding this particular type of SERS nanoparticle is provided in WO 2006/073439, U.S. Pat. No. 6,514,767, and U.S. patent application Ser. No. 60/557,729, each of which are incorporated herein by reference as they pertain to the detailed description of each application or patent and as they relate to SERS nanoparticles and SACNs.

In an embodiment, one type of SERS nanoparticle includes Surface Enhanced Spectroscopy-Active Composite Nanoparticles (SACNs). SACNs and methods of making SACNs are described in WO 2006/073439, U.S. Pat. No. 6,514,767, and U.S. patent application Ser. No. 60/557,729, each of which is incorporated herein by reference as they pertain to the detailed description of each application or patent and as they relate to SACNs. Embodiments of the SACNs can include a SERS nanoparticle, a submonolayer, monolayer, or multilayer of reporter molecules in close proximity to the metal surface, and an encapsulating shell (e.g., a polymer, glass (SiOₓ), or other dielectric material). In an embodiment, the reporter compound is disposed at the interface between the SERS nanoparticle and the encapsulant. In an embodiment, a SACN comprises (i) a metal nanoparticle core (e.g., Au or Ag), (ii) a Raman-active reporter (reporter compound), that gives a unique vibrational signature, and (iii) an SiOₓ: encapsulant that “locks” the reporter molecules in place while also providing a highly compatible surface for subsequent immobilization of biomolecules. The glass coating can also stabilize the particles against aggregation and can prevent competitive adsorption of unwanted species. In an embodiment, the SERS nanoparticles are comprised of polymer coatings adjacent to the nanoparticle.

As used herein, the term “reporter compound” includes Raman-active compounds that produce a unique SERS signature in response to excitation by a laser. In certain embodiments, Raman-active organic compounds are polycyclic aromatic or heteroaromatic compounds. In an embodiment, the reporter compound can include, but is not limited to, 4-mercaptopyridine (4-MP); trans-4,4′bis(pyridyl)ethylene (BPE); quinolinethiol; 4,4′-dipyridyl, 1,4-phenyldiisocyanide; mercaptobenzamidazole; 4-cyanopyridine; 1′,3,3,3′,3′-hexamethylindotricarbocyanine iodide; 3,3′-diethyltiatricarbocyanine; malachite green isothiocyanate; bis-(pyridyl)acetylenes; Bodipy; TRIT (tetramethyl rhodamine isothiol); NBD (7-nitrobenz-2-oxa-1,3-diazole); Texas Red dye; phthalic acid; terephthalic acid; isophthalic acid; cresyl fast violet; cresyl blue violet; brilliant cresyl blue; para-aminobenzoic acid; erythrosine; biotin; digoxigenin; 5-carboxy-4′,5′-dichloro-2′,7′-dimethoxy fluorescein; 5-carboxy-2′,4′,5′,7′-tetrachlorofluorescein; 5-carboxyfluorescein; 5-carboxy rhodamine; 6-carboxyrhodamine; 6-carboxyletramethyl amino phthalocyanines; azomethines; cyanines; xanthines; succinylfluoresceins; aminoacridine; fullerenes; organocyanides (e.g., isocyanide), methylene blue indigo carmine, and indocyanine green (ICG), and the like, and combinations thereof.

A COIN includes several fused or aggregated primary metal crystal particles with the Raman-active organic compounds (reporter compound) adsorbed on the surface, within the junctions of the primary particles, or embedded in the crystal lattice of the primary metal particles. The primary metal crystal particles are about 15 nm to 30 nm, while the fused or aggregated COIN is about 50 nm to about 200 nm. The primary metal crystal particle is made of materials such as, but not limited to, gold, silver, platinum copper aluminum, and the like. The Raman-active organic compound refers to an organic molecule that produces a unique SERS signature in response to excitation by a laser. Additional details regarding COINS are described in U.S. patent application Nos. 2005/0142567, 2006/0234248, and 2007/0048746, each of which is incorporated herein by reference for the corresponding discussion.

COINs can also serve as Raman nanoparticles to provide imaging signals. The COINs can be functionalized so they have better solubility in blood and can target potential targets in a living subject. Multiple COINs can be used with other Raman nanoparticles in order to provide multiplexing of signals.

In an embodiment, the Raman agent can be incorporated (e.g., disposed inside and/or attached to the surface of) or encapsulated into a biological agent (e.g., a cell or a virus). In particular, the Raman agent can be incorporated into stem cells, t-cells, bacterial strains, Red blood cells, white blood cells, and the like. As the encapsulating virus, bacteria, or stem cell moves through the body or within an area, the Raman imaging system can be used to monitor/track the virus, bacteria, or cell. Studying cell motility and tracking its natural distribution in the body is an important biological process that can offer scientists important information on how to better design diagnostics and therapeutics. By using a stem cell, for instance, incorporating a Raman agent (e.g. Raman active dyes or Raman nanoparticles) one could use the Raman signal to monitor its localization within the body after it has been administered for therapy for instance. One could also study the homing effects that bacteria, viruses, t-cells, or even macrophages have on tumor sites if these cells were to be previously encapsulated with Raman agents (e.g. Raman dyes or Raman nanoparticles). One could essentially use their Raman active signal as a reporter to track where exactly these cellular entities have localized after administration.

In an embodiment, the method of monitoring biological agent includes introducing a first type of biological agent that includes a first type of Raman agent to a sample or a subject. After an appropriate amount of time, a Raman imaging device can be positioned in the area of interest (e.g., area of the colon, or the entire colon, or the like) so that the area can be imaged (e.g., in front of the Raman imaging device, behind the imaging device, or near the tip of the Raman imaging device). In an embodiment the Raman imaging device can be positioned adjacent an area that may include the biological agent. Subsequently, the area is exposed to a light beam, where if the biological agent including a Raman agent is present, the light beam is scattered. The light beam that is scattered is referred to as a Raman scattered light energy. The Raman scattered light can be detected using the Raman imaging device at various positions relative to the area. The detection of the Raman scattered light indicates that the biological agent is present in the area. If multiple biological agents or types of biological agents are introduced, each can include the same type of Raman agent or different types of Raman agents. If different types of Raman agents are used, then the type and/or amount of the biological agent can be determined based on the type of Raman agent detected. As described herein the area can be circumferentially scanned, mapped, and the like.

In an embodiment, the Raman compounds can include a reporter compound as noted above conjugated to a targeting ligand, so that the Raman agent or compound can have an affinity for a targeting ligand.

In an embodiment, the Raman agent can include a targeting ligand that is a chemical or biological ligand or compound having an affinity for one or more targets (e.g., also referred to as a “specific target” or “targeted area”). In an embodiment, the targeting ligand can include, but is not limited to, a drug, a therapeutic agent, a radiological agent, a chemological agent, a small molecule drug, a biological agent (e.g., antibodies, peptides, proteins, apatamers, antigens, and the like) and combinations thereof, that has an affinity for a target or a related biological event corresponding to the target. It should be noted that Raman agent modified with conjugation to other molecules (e.g., antibodies, proteins, peptides, apatamers, small molecules, and the like) in order to target the Raman agent to a particular molecular target are intended to be covered by embodiments of the present disclosure. For example, a Raman agent can be modified with a peptide so that it can target new blood vessels in tumors or a chemical associated with a specific cancer, tumor, or precancerous tissue. In an embodiment, the targeting ligand can have an affinity for a target such as cancer, tumor, precancerous cells or tissue, atherosclerosis, fibrosis. In another embodiment, the targeting ligand can be used for trafficking (where the Raman agent is incorporated into viruses or cells (e.g., stem cells, t-cells, Red blood cells, white blood cells, and the like)) to look at distribution in the body.

In an embodiment, a first type of Raman agent and a second type of Raman agent can be given to a subject to locate and detect the presence of an adenoma. The first type of Raman agent has an affinity for tubular adenomas and the second type of Raman agent has an affinity for villous adenomas, where if both types of agents are present in a certain ratio, then tubulovillis adenoma is present at the area.

Embodiments of the present disclosure include methods of using a Raman imaging device (e.g., Raman endoscope probe) in conjunction with one or more types of Raman agents to image, detect, study, monitor, evaluate, and/or screen a subject (e.g., whole-body or a portion thereof (e.g., bronchioles, esophagus, colon, rectum, skin, oral mucosa, intraoperatively any organ, and the like)). The Raman agent(s) is administered to the subject and then the subject (e.g., a portion such as the colon and the like) can be imaged using an endoscope including a Raman imaging device. In an embodiment, the Raman imaging system can just be used to measure a signal, where the signal originated from a particular location. In an embodiment, the Raman imaging device, in conjunction with an analysis system (e.g., computer, software, etc, are interfaced with the Raman imaging device), is capable of creating an image of an examined area of a living host (e.g., colon), which is in contrast to just measuring a signal in a host.

The following describes an embodiment using a Raman endoscope probe and a subject is administered one or more Raman agents. An endoscope including the Raman endoscope probe is introduced to the subject (e.g., endoscopically, laproscopically, intraoperatively, or surgically). The introduction can be via an orifice or through a surgical incision. The endoscope including the Raman endoscope probe can be moved to scan an area or if the specific target area is known, the endoscope can be moved adjacent the specific target area. Depending on the type of Raman endoscope probe (e.g., forward view or side view), the position (e.g., in front of area, behind the area, or area at the tip of the probe) of the endoscope can be varied to obtain the optimum scattered light energy from the Raman agent(s). The Raman endoscope can be used to scan an area and/or map an area in the subject.

A Raman image (e.g., the Raman scattered light energy) using embodiments of the present disclosure is different from a bulk signal in that the Raman image is a visual representation of signal as a function of location (e.g., a particular location in the host such as a part (e.g., a few millimeters, a centimeter or more) of the colon or the like).

Embodiments of the present disclosure can be used to map an area. The area can include a portion or the entire area of the: cervix, bladder, bronchioles, esophagus, stomach, colon, rectum, skin, oral mucosa, and intraoperatively or laparoscopically an organ. In an embodiment, the mapping can be conducted by exposing the area to the Raman imaging device by moving the Raman imaging device. An area can be mapped prior to and/or after introducing one or more types of Raman agents and/or one or more types of biological agents to the subject or sample. The Raman imaging device detects the Raman scattered light and this can be correlated to a position in the area so that a map can be obtained for the area. In an embodiment, the area can be monitored as a function of time and can be used to determine the impact of a particular treatment or the like.

Embodiments of the present disclosure include administering or otherwise introducing one or more types of Raman agents (e.g., have emissions at different wavelengths, or two different types of Raman agents) to a subject. In an embodiment, the Raman agents are introduced by disposing the Raman agents on the tissue and then washing the tissue to remove unattached Raman agents. In embodiments including two or more different types of Raman agents, each of the Raman agents has a different Raman signature and/or can be directed to different targets. Subsequently, the subject can be imaged using a Raman endoscope probe via the introduction of an endoscope to the subject. In an embodiment, the different Raman agents used in conjunction with the Raman endoscope probe could be used to image different portions (e.g., tissue, cells, organs, and the like) of the subject and/or detect different types of targets. Also nonspecific Raman agents can be used to normalize the background signal that may be caused by non-uniform dispersal of the Raman agent and/or non-uniform washing of the area.

In another embodiment, each of the different Raman agents could be directed to different biological targets relating to the same disease, condition, or related biological event. In this embodiment, the different types of Raman agents could be used to determine the presence or absence of one or more features of the disease, condition, or related biological event, which is useful for certain cancers and their progression over time and even after treatment to look at their response to therapy (e.g., the type or severity of a cancer can be determined by the presence of one or two targets, and treatment is based on the type or severity of the cancer). Embodiments of the present disclosure include other ways in which a combination of Raman agents could be used in embodiments of the present disclosure.

In another embodiment of the present disclosure, the Raman endoscope probe and the Raman agents can be combined with an anatomical image and/or a functional image of the same subject generated from an anatomical imaging system. The anatomical imaging system can include, but is not limited to, bright field white light imaging, computer topography (CT), ultrasound, magnetic resonance imaging (MRI), and the like. The combination of multiple functional images or a functional image with an anatomical image would provide more useful information about the exact location of a specific molecular event. The anatomy would tell where, and the molecular image (functional image) would tell how much molecular signal from a given anatomical coordinate.

In each of the embodiments described above and herein, one or more types of untargeted Raman agents can be used in addition to the targeted Raman agent(s). The use of the untargeted Raman agents allows for an assessment of the ratio between or among specific binding to non-specific binding Raman agents, and thus providing a ratiometric estimate of truly bound Raman agent(s). The untargeted Raman agents can be used to compare areas where the targeted Raman agents are located (e.g., the targeted area or specific target) to the areas where the targeted Raman agents are not located. The use of the untargeted areas can provide a baseline that can be used in the analysis, evaluation, and/or mapping of an area or targeted area.

It should be noted that the amount effective to result in uptake of a Raman agent into the cells or tissue of the subject depends upon a variety of factors, including for example, the age, body weight, general health, sex, and diet of the host; the time of administration; the route of administration; the rate of excretion of the specific compound employed; the duration of the treatment; the existence of other drugs used in combination or coincidental with the specific composition employed; and like factors well known in the medical arts.

Embodiments of the present disclosure can also be used to identify the surgical margins for a tumor resection. In particular, a surgeon can use the imaging information provided by embodiments of the present disclosure to guide surgery. Embodiments of the present disclosure can be used in-situ morphological mapping, in particular, to map cancer tissue to guide therapy. Embodiments of the present disclosure can be used to develop an understanding of the morphological composition of a tumor at the molecular level and optimize their therapies accordingly. Embodiments of the present disclosure can also be used targeted thermal ablation. The therapy could take advantage of the energy-absorbing properties and the targeting properties of the nanoparticles to thermally ablate tumor cells.

### Kits

The present disclosure also provides packaged pharmaceutical compositions comprising a pharmaceutically acceptable carrier and one or more Raman agents and a Raman imaging device such as a Raman endoscope probe or handheld Raman device. Other packaged pharmaceutical compositions provided by the present disclosure further include indicia including at least one of: instructions for using the Raman imaging device and the Raman agent to image a subject.

This disclosure encompasses kits that include, but are not limited to, Raman agents and a Raman imaging device and directions (written instructions for their use). The Raman agent can be tailored to the particular biological event to be monitored as described herein. The kit can further include appropriate buffers and reagents known in the art for administering the Raman agent to the subject. The Raman agent and carrier may be provided in solution or in lyophilized form. When Raman agent and carrier of the kit are in lyophilized form, the kit may optionally contain a sterile and physiologically acceptable reconstitution medium such as water, saline, buffered saline, and the like.

### EXAMPLES

Now having described the embodiments of the present disclosure, in general, the example describes some additional embodiments of the present disclosure. While embodiments of the present disclosure are described in connection with the example and the corresponding text and figures, there is no intent to limit embodiments of the present disclosure to these descriptions. On the contrary, the intent is to cover all alternatives, modifications, and equivalents included within the spirit and scope of embodiments of the present disclosure.

### Introduction:

Early detection remains one of the most powerful ways to improve prognosis for cancer patients. As a result, a significant effort has been made to develop new diagnostic strategies to detect early stage cancer more sensitively, both in-vitro and in-vivo. Raman spectroscopy has proven to be a powerful analytical tool that offers unsurpassed sensitivity and multiplexing capabilities. Harnessing these unique properties for early detection of cancer could serve as a powerful diagnostic strategy, with the potential to significantly impact the survival rate of those patients diagnosed earlier.

Raman spectroscopy is based on an inelastic light scattering phenomenon that can offer detailed chemical information, but occurs very infrequently. Most photons are elastically scattered when they interact with matter, where the scattered photons maintain the same energy and wavelength as the incident photons. However, a small fraction of light, approximately 1 in 10 million photons, is inelastically scattered, meaning the scattered photons lose energy resulting in a longer wavelength. This inelastic scattering of light was first observed in 1928 by C. V. Raman and is termed the Raman Effect. Since then, Raman spectroscopy has been predominantly used as an analytical tool to determine the molecular composition of materials based on the energy differences seen between the incident and scattered photons. However, more recently, researchers have used Raman spectroscopy to interrogate various biomedical processes including analysis of cell populations, excised tissue samples, preclinical animal models and even clinical diagnosis.

Researchers have attempted to utilize Raman spectroscopy for clinical diagnosis by interrogating the intrinsic chemical differences between malignant and normal tissues. However the weak effect associated with intrinsic Raman scattering remains a problem, leading to long exposure times, poor signal, and, as a result, suboptimal sensitivity. The two main challenges that often make it difficult to translate Raman spectroscopy to the clinic include its limited depth of penetration and its intrinsically weak effect, only producing 1 inellastically scattered photon for every 10 million elastically scattered photons.

Endoscopy has become an important tool in visually assessing structural details deep within the body. It allows physicians to take a close-up look at various tissues and organs with a minimally invasive outpatient procedure. Several organs are easily accessible with endoscopic tools, including the bladder (cystoscopy), cervix (colposcopy), lung (bronchoscopy), esophagus and stomach (upper GI endoscopy), and most notably the colon (colonoscopy). It is well known that screening and treatment of polyps via endoscopy could prevent the majority of colorectal cancers (−80%), and as a result significantly decrease the mortality rate due to colorectal cancers. However, white-light endoscopy alone is an imperfect technology that only offers structural details based on visual observation and can result in miss rates of up to 25%. It was also reported that flat lesions in the colon, which are more difficult to detect with white light endoscopy, were five times more likely to contain cancerous tissue than the visually apparent polyps detected by conventional colonoscopy. This problem of failed detection could be significantly minimized with the addition of a molecular imaging component that offers important functional information in conjunction with the structural based white light endoscopes used today.

### Discussion:

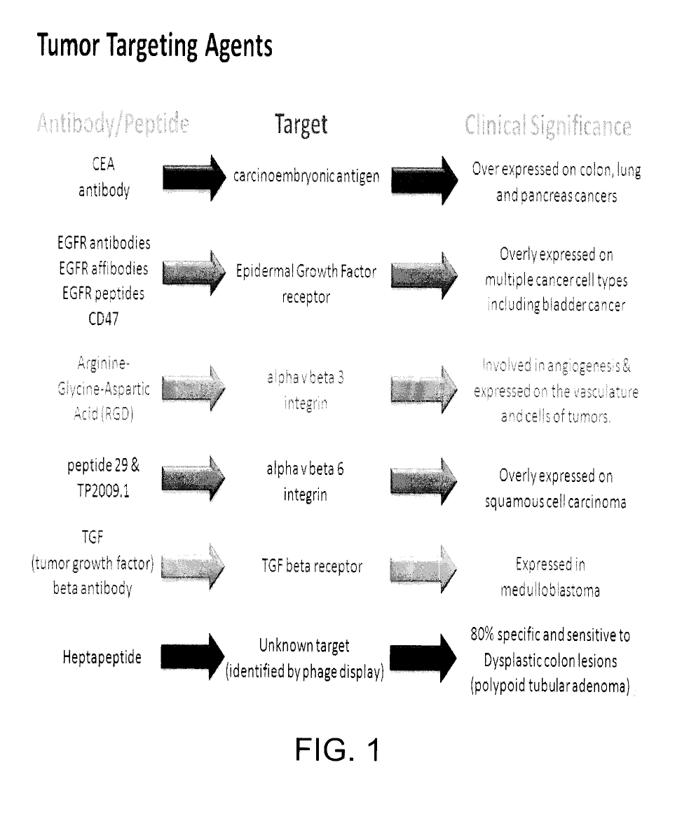

The present disclosure describes the use of surface enhanced Raman scattering (SERS) nanoparticles as tumor targeting contrast agents. These gold based nanoparticles exhibit a dramatic increase in the Raman scattered light they emit due to a plasmon resonance effect on their metallic surface. Various small molecules are adsorbed onto this nanoroughened metallic surface and once light interacts with these small molecules an increased Raman Effect is observed (up to several orders of magnitude more). The SERS nanoparticles can be coated with one or more types of tumor targeting agent as shown in the FIG. 1.

Disclosed here is a small flexible fiber optic based Raman device (e.g., diameter=5.5 mm). This device has been carefully designed to be sent through the accessory channel of a clinical endoscope to sensitively detect these tumor targeted SERS nanoparticles while overcoming the limited depth of penetration associated with most optical techniques. Physicians will now have the ability to utilize the unique functional information Raman spectroscopy has to offer during endoscopic, laparoscopic or surgical procedures.

In an embodiment, the setup can include a laser (e.g., continuous wave at 785 nm) coupled into single-mode fiber, which is ultimately used to illuminate a Raman-active sample. In an embodiment, a single lens is used to both collimate the illuminating beam and for collecting the SERS scattered light into a multi-mode fiber bundle. The bundle can include 36 multi-mode fibers, for example, surrounding the single-mode fiber for in-line illumination and light collection. At the proximal end of the fiber bundle, the multi-mode fibers can be arranged into a linear array and coupled into a spectrometer. The spectrometer disperses the wavelengths of light collected by the multimode fiber onto a cooled, deep-depletion, charge-coupled-device (CCD) camera. The camera electronics perform full vertical binning of the sensor array in order to sum the spectral intensity of the Raman signal at each wavelength. Once a spectral acquisition is obtained for a given exposure time, the acquired signal is unmixed using a library of reference measurements—nanoparticle signatures and background signals—and a direct-classical-least-squares fitting algorithm. The unmixing process reveals the relative concentrations of various nanoparticles within the target sample.

### Method Description:

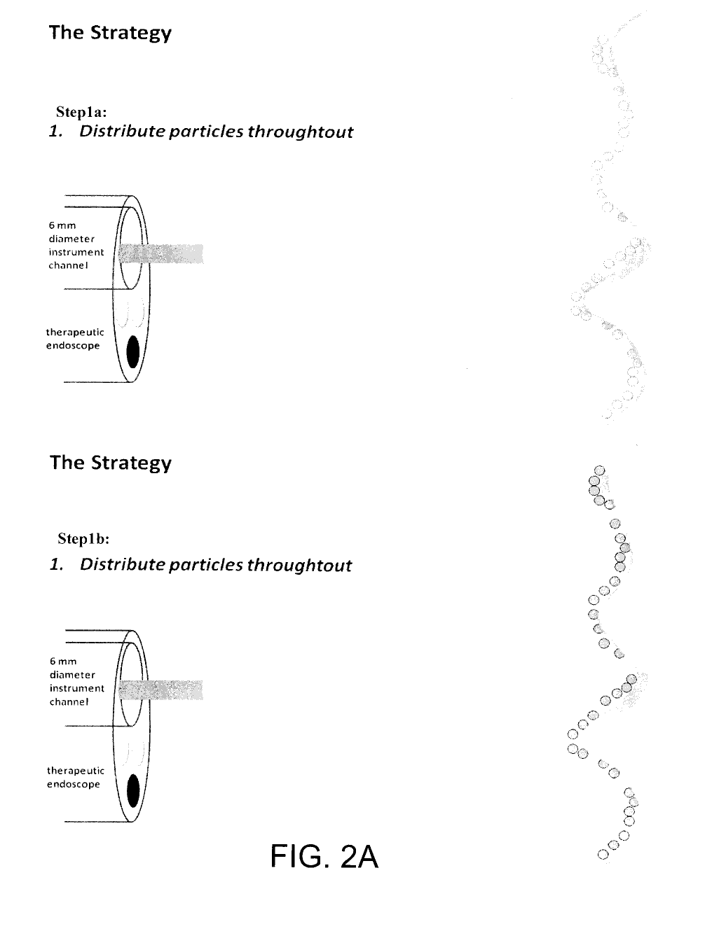

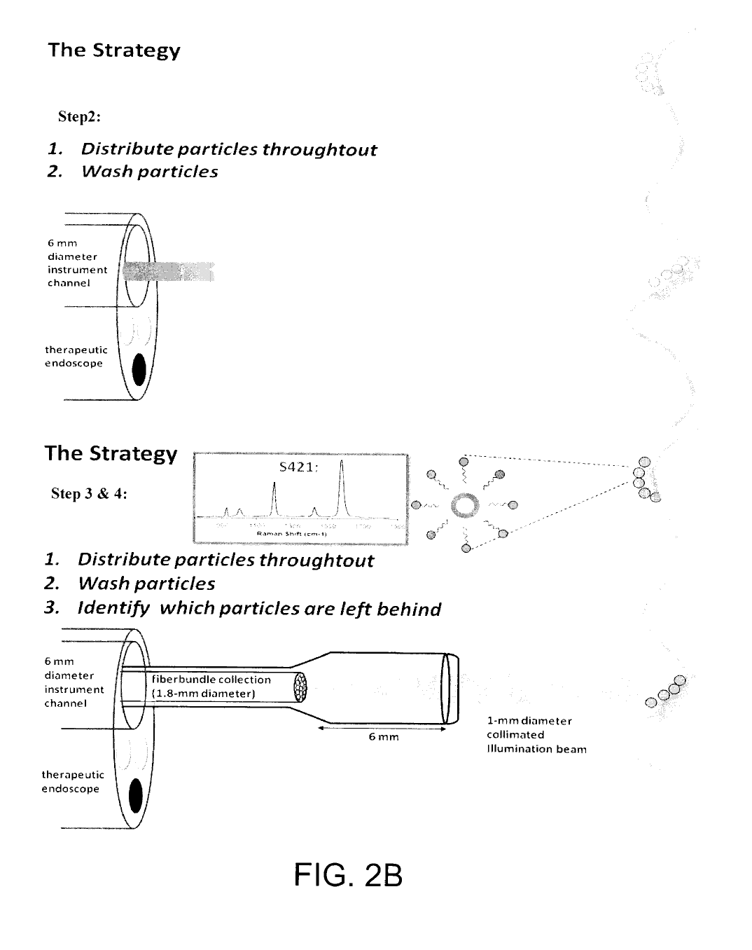

In an embodiment, the device can be used in the following way: 1. The physician topically distributes a single or multiplexed panel of tumor-targeting surface-enhanced-Raman-scattering (SERS) nanoparticles uniformly within the colon. 2. The physician washes the colon, leaving behind only specific targeted SERS nanoparticles that have bound to the tumor. 3. The Raman-endoscopic device is inserted into the working channel of an endoscope in order to detect and quantify the presence of a single or multiplexed panel of tumor-targeting SERS nanoparticles. 4. The physician interprets the pathological condition of interest based on the identified location and type of the bound SERS nanoparticles. This process is depicted in FIGS. 2A and 2B.

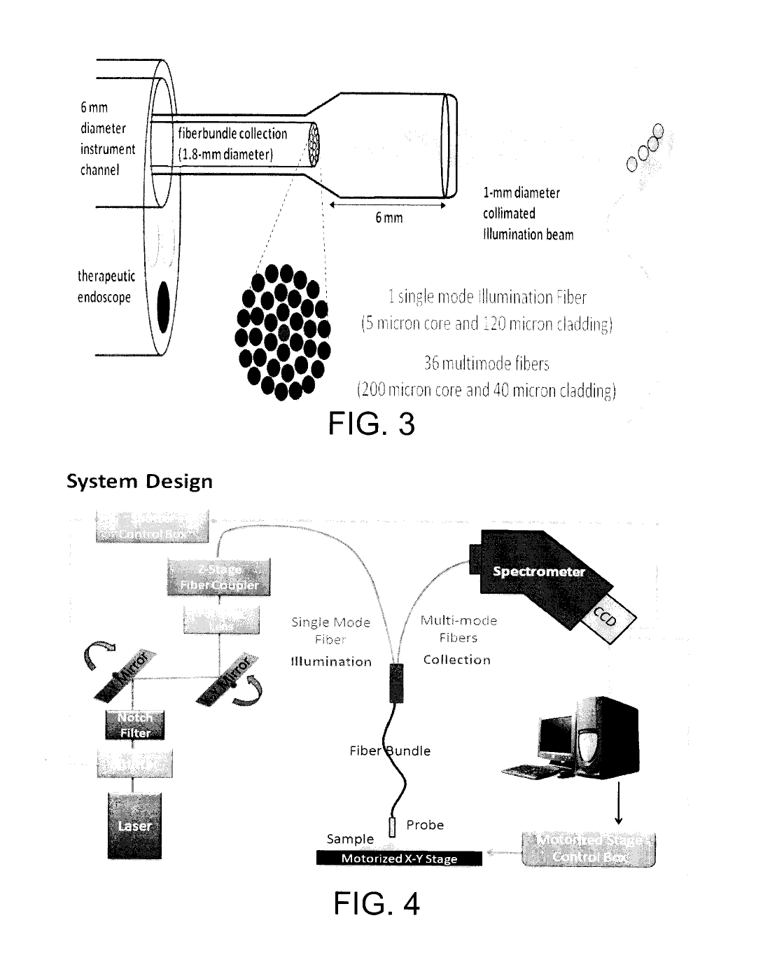

In an embodiment shown in FIG. 3, the Raman endoscope can be designed to be inserted through the accessory channel of a clinical endoscope with a 6 mm instrument channel. In an embodiment, the Raman endoscope is comprised of 1 single mode illumination fiber that is surrounded by a bundle of 36 multimode collection fibers totaling a diameter of 1.8 mm. The excitation laser light is collimated by a lens to emit an illumination spot size of ˜1.2 mm. The circles represent the SERS nanoparticles. The light gray surface on the right is an illustration of the colon wall. The dark gray region on the colon wall represents a flat cancerous lesion. The illustration depicts that the SERS nanoparticles preferentially bind to cancerous tissue, which for this illustration is a flat lesion.

An embodiment of the system is shown in FIG. 4. The 785 nm laser can be controlled by a shutter driven by a computer driven shutter controller. The laser is then passed through a notch filter which ensures a narrow 785 nm bandwidth, guided through a series of mirrors and refocused to a single mode fiber to illuminate a sample. The light collected by the multi-mode fibers is dispersed by wavelengths onto a CCD via a spectrometer.

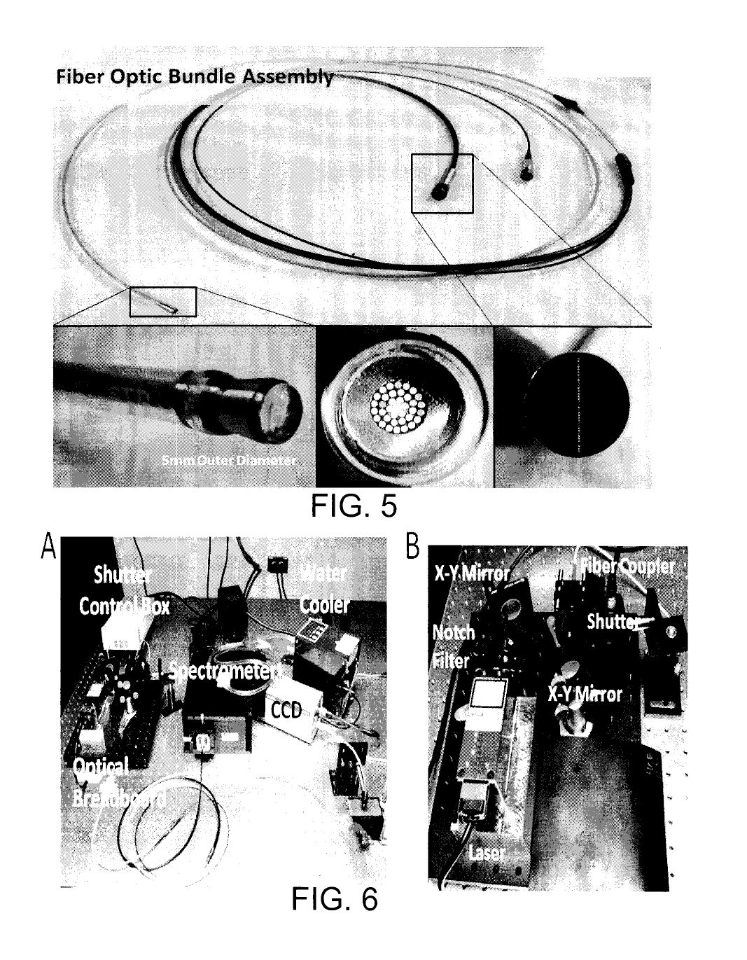

The photograph FIG. 5 depicts the final fabricated Raman endoscope to be used for clinical studies. The bottom panel shows an enlarged digital photograph of the endoscope head (left), a magnified photograph of the fiber bundle (middle) and the back end of the device (right) that shows a linear array of the 36 collection fibers that are specially aligned to fit into a spectrometer.

FIG. 6A shows the optical system. The optical breadboard couples the laser beam into a single mode fiber of the fiber bundle. The proximal end of the fiber bundle is coupled into the spectrometer and a deep depleted CCD is used to image the dispersed light. The CCD is water cooled to minimize dark current. FIG. 6B shows the details of the optical breadboard from FIG. 6A are shown here. The arrows show the path traveled by the beam. The laser is continuous-wave at 785 nm. The notch filter ensures that the full-width half max is no more than 3 nm. Two x-y mirrors are used help couple the laser into a single mode fiber. The fiber coupler includes a focusing lens mounted onto a linear stage, which is also used for alignment of the beam into the single mode fiber. Prior to the CCD acquiring a signal, the CCD sends a signal to the shutter control box which then opens the shutter. The shutter is nominally in the closed position.

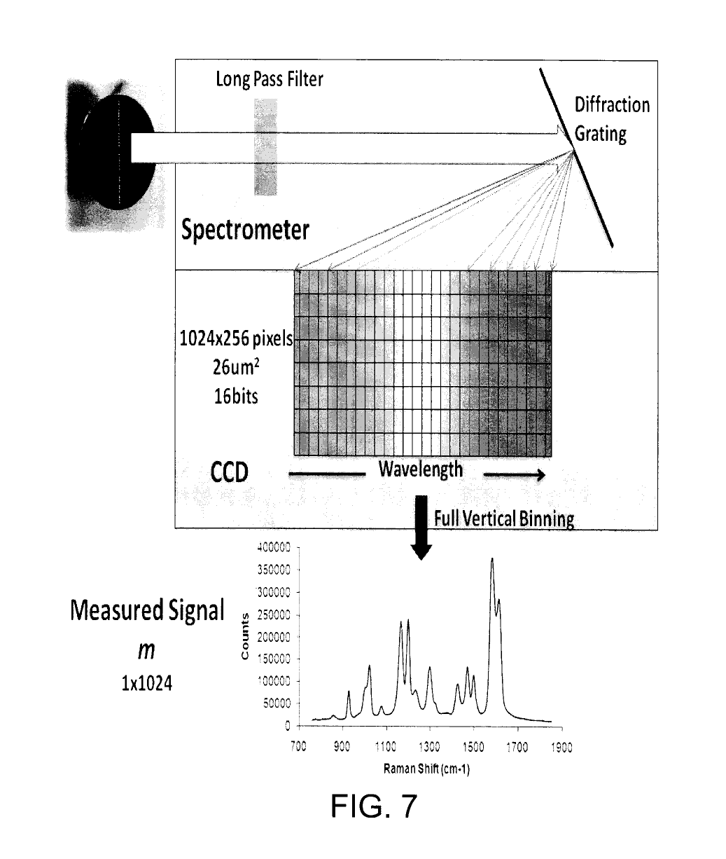

The spectrometer horizontally disperses the light collected by the multimode fiber and is imaged onto a cooled, deep-depletion charge coupled device (CCD) array (See FIG. 7). The CCD then performs a full vertical binning (FVB) of the sensor array in order to sum the spectral intensity of the multi-mode fibers for a given discrete delta in wavelength. Once a FVB acquisition is obtained for a given delta of time the acquired signal is decomposed to a weighting factor, using the direct classical least squares (DCLS) algorithm, which represents the relative intensity of the acquired signal to a known spectral signature or combination of known spectral signatures of the Raman nanoparticles.

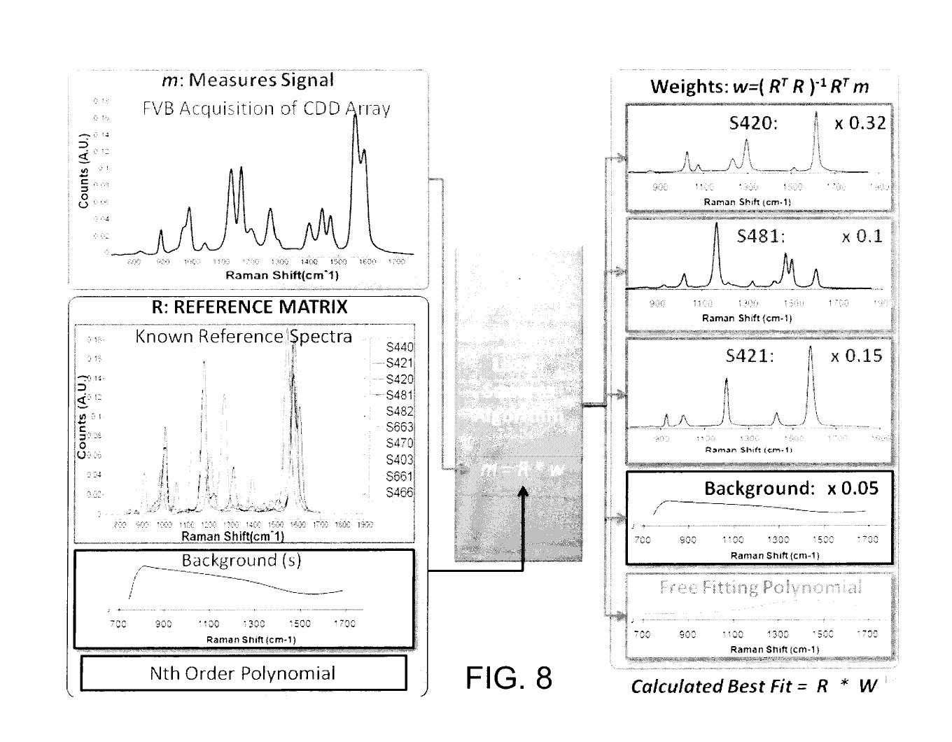

In order to accomplish unmixing, the direct classical least-squares (DCLS) method, also called the linear unmixing method, was utilized (See FIG. 8). This algorithm compares the acquired signal with known reference spectra, representative background spectra, and a freely-varying polynomial component. The representative background spectrum is included as a reference spectrum in our analysis, and it is free to vary in magnitude for each measurement. The output of the DCLS algorithm is a magnitude of its extracted spectrum relative to its respective reference spectrum. The SERS nanotags are comprised of a 60-nm-diameter Au core coated with a monolayer of Raman-active organic molecules and encapsulated with a 30-nm-diameter silica shell, making the entire particle on the order of about 120 nm.

Characterization of Raman endoscope performance.

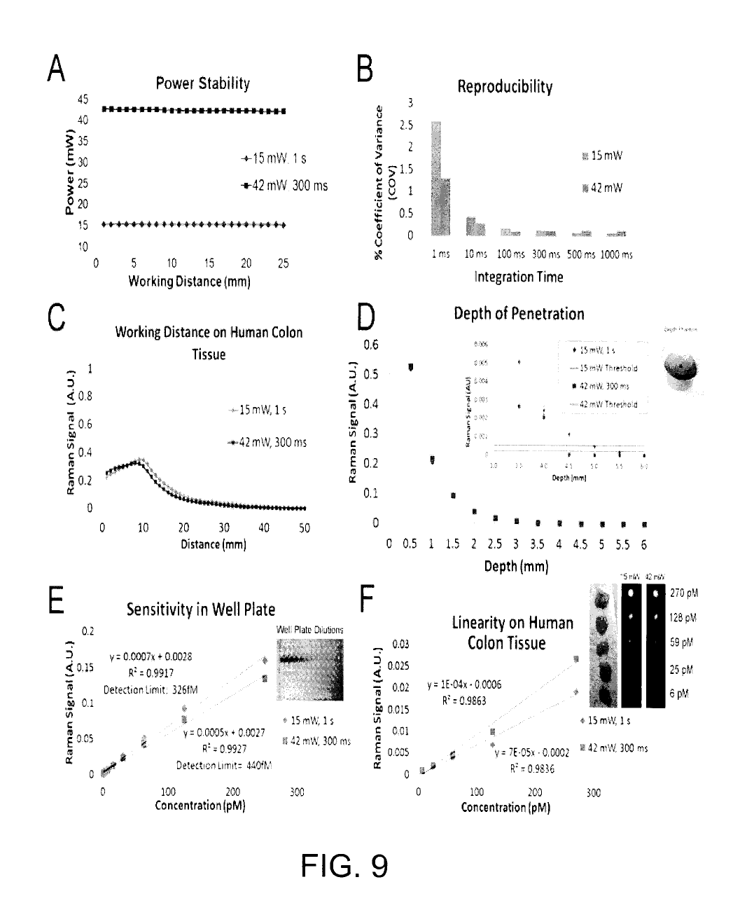

FIG. 9A illustrates a graph depicting stable power output over a working distance of 25 mm away from sample surface.

FIG. 9B illustrates a graph showing excellent Raman signal reproducibility of our Raman endoscope over several integration times. Notice how shorter integration times lead to more variability in signal, however even at 1 ms integration times the reproducibility is still good with only a COV of 2.5%.

FIG. 9C illustrates a graph showing stable Raman signal over a working distance of 10 mm away from sample surface. Notice that the Raman signal drops off significantly (1/distance²) after the Raman endoscope is more than 10 mm away from the sample.

FIG. 9D is a graph showing that our Raman endoscope is able to detect Raman signal at depths of 4-5 mm when using 1 sec integration times in our tissue mimicking phantom (photo at right).

FIG. 9E is a graph showing the sensitivity of our Raman endoscope where the limit of detection was 326 fM (15 mW at 1 s) and 440 fM (42 mW at 300 ms) of SERS nanoparticles in a well plate (photo at right). Notice how SERS Raman concentration correlates linearly with Raman signal at both laser powers.

FIG. 9F is a graph depicting the sensitivity of our Raman endoscope after topically applying diluted concentration of SERS nanoparticles onto fresh human colon tissue samples (photo at right). Raman signal linearly correlates with the concentration of SERS nanoparticles applied.

Demonstration of multiplexing on quartz slide.

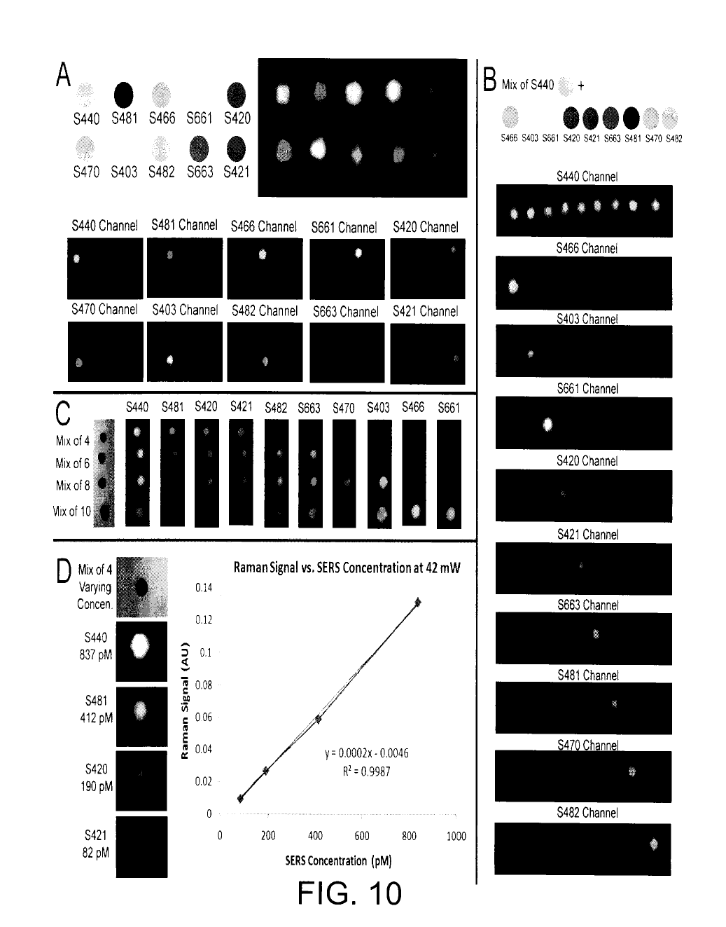

FIG. 10A illustrates ten unique flavors of SERS nanoparticles spatially separated onto a piece of quartz. The Raman map acquired identifies all 10 flavors correctly. Notice how each of the flavors are correctly represented in each of the SERS nanoparticle channels in the panels below.

FIG. 10B illustrates an equal mixture of S440 and one other flavor is placed in separate drops across a piece of quartz to characterize dual colocalization of SERS nanoparticles. The Raman map correctly identifies the presence of each of the two flavors of SERS nanoparticles in each mixed droplet as shown in the separate SERS channels below.

FIG. 10C illustrates a demonstration of multiple colocalized SERS flavors including mixtures of 4, 6, 8 and all 10 SERS nanoparticles within the same droplet on quartz. The Raman maps shown depict the correct location of each of the SERS flavors within each of the mixtures.

FIG. 10D illustrates a mix of 4 SERS nanoparticle flavors each at varying concentrations. The post processing software was able to spectrally separate each flavor into its respective channel correctly and the Raman maps to the left show a decrease in Raman intensity that correlates with the concentrations of each of the SERS flavors. The graph to the right depicts a linear correlation between the SERS concentration of each flavor and the Raman signal with an R² value of 0.9987.

Demonstration of multiplexing on human tissue.

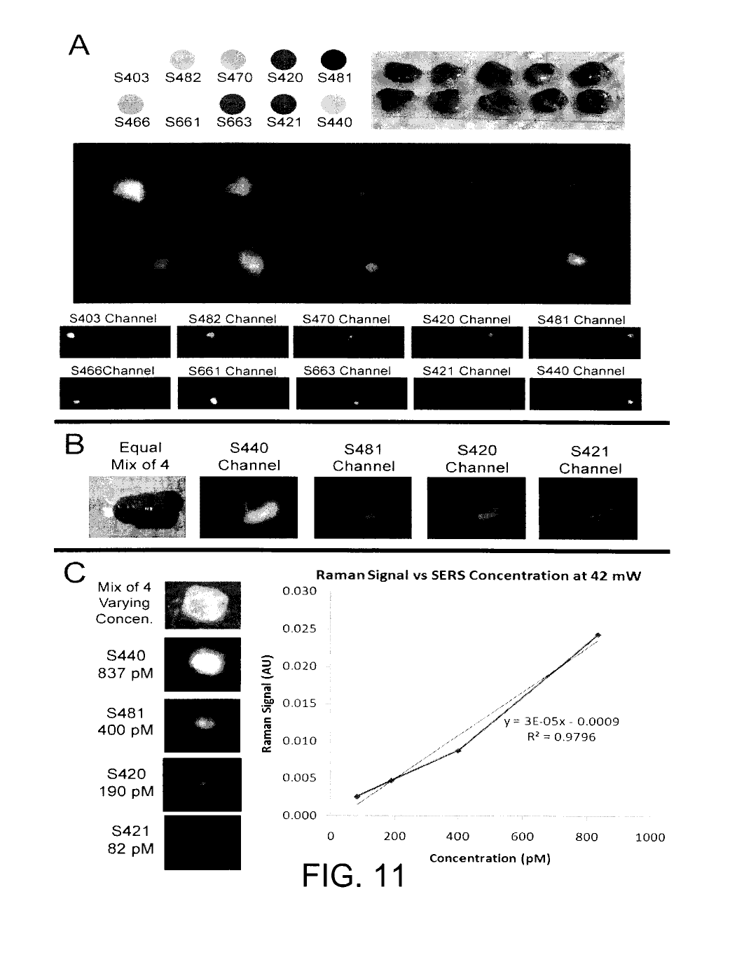

FIG. 11A illustrates ten unique flavors of SERS nanoparticles spatially separated onto 10 separate pieces of fresh human colon tissue. The Raman map acquired identifies all 10 flavors correctly. Notice how each of the flavors are correctly represented in each of the SERS nanoparticle channels in the panels below.

FIG. 11B illustrates a demonstration of colocalized multiplexing, where 4 SERS flavors were equally mixed and applied on a single piece of human colon tissue. The post processing software was able to spectrally separate each flavor into its respective channel correctly as shown in the Raman maps to the right.

FIG. 11C illustrates a mix of 4 SERS nanoparticle flavors each at varying concentrations were mixed together and applied to a single piece of human colon tissue. The post processing software was able to spectrally separate each flavor into its respective channel correctly and the Raman maps at the left show a decrease in Raman intensity that correlates with the concentrations of each of the SERS flavors. The graph to the right depicts a linear correlation between the SERS concentration of each flavor and the Raman signal with an R² value of 0.9796.

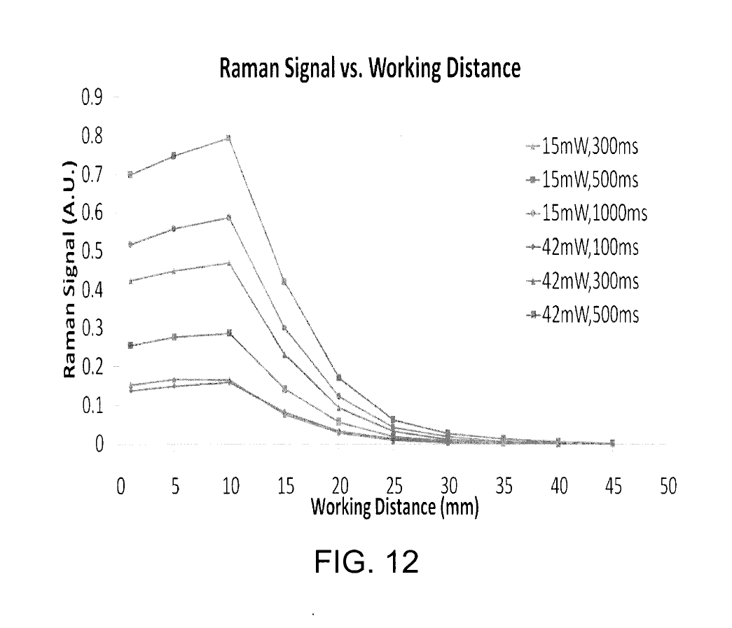

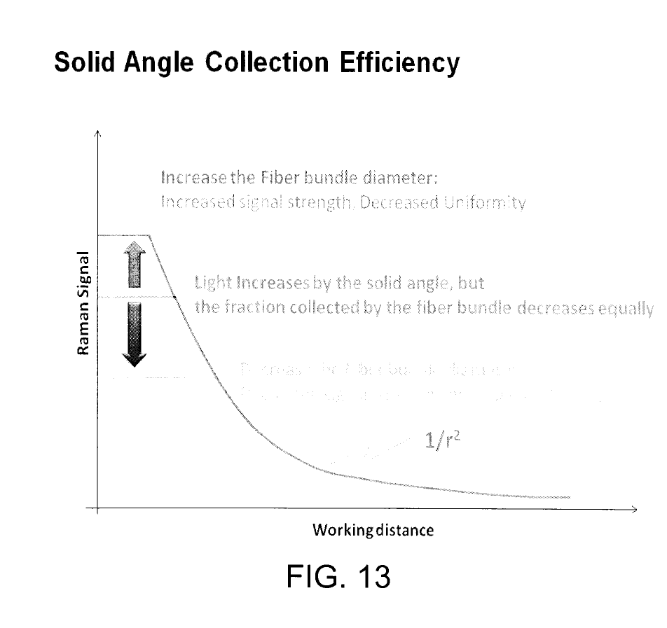

As shown FIG. 12, the Raman signal was evaluated over a variety of working distances from 1 mm to 45 mm and for a variety of power and integration times. The sample used was a 3 μL drop containing 16 nM of S440 that was place on a quartz slide. The Raman signal is consistent for working distances of up to 1 cm; after which the signal drop as 1/d², where d is the working distance (See FIG. 13). The drop off in signal is due to the solid angle collection efficiency.

Raman signal vs. laser power.

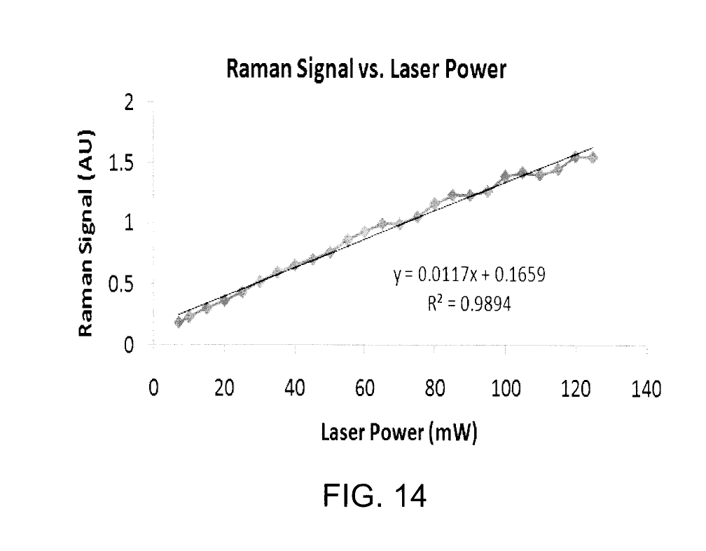

The graph in FIG. 14 depicts a linear trend where Raman signal increases linearly with increased laser power settings.

Clinical application and utility of Raman endoscope in humans.

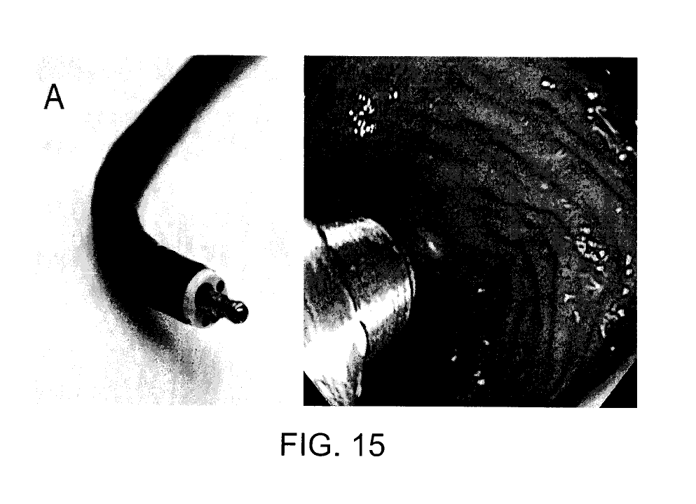

FIG. 15A illustrates an embodiment of a Raman endoscope inserted into the instrument channel of a conventional clinical colonoscope.

FIG. 15B illustrates a magnified digital photograph taken from the white light ndoscopy component of the clinical colonoscope portraying our Raman endoscope protruding from the instrument channel and illuminating a spot on the colon wall in a human patient.

### Signal Analysis.

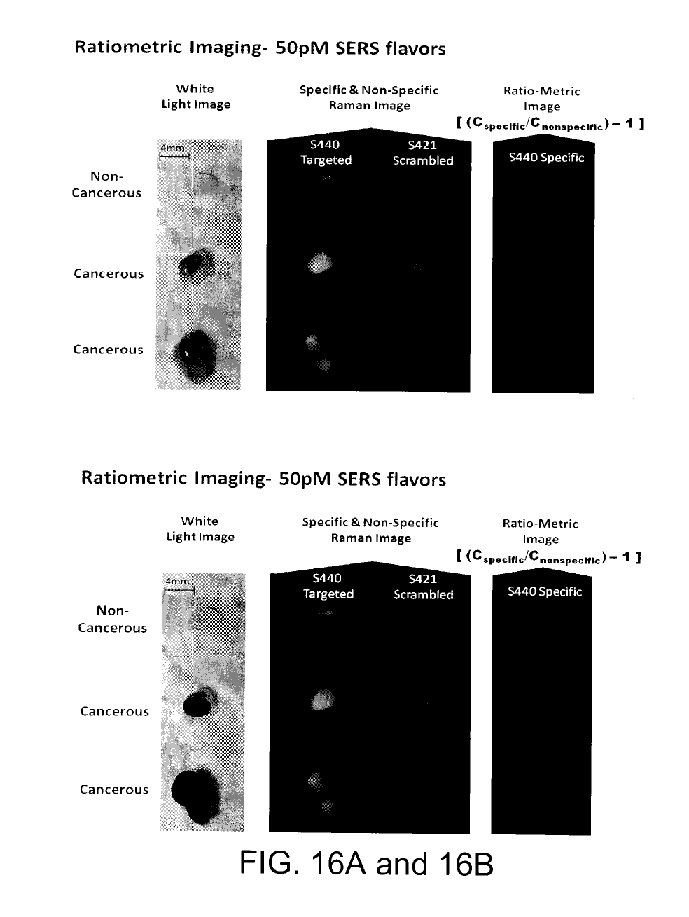

There are challenges with rinsing of the target specific Raman nanoparticles because it can be non-uniform and inefficient especially in thick tissue such as the colon which has many folds. As a result, it can be difficult to distinguish between bound and unbound Raman nanoparticles. This can lead to ambiguous contrast and a large noisy background response, due to unbound probes, that may obscure the bound tissue specific targeted Raman particles. Since the application of Raman nanoparticles, as well as the rinsing away of unbound probes, is neither efficient nor uniform in fresh intact tissue, a large nonspecific background often exists that is unrelated to the target of interest. The technique we have developed utilizes two varieties of Raman nanoparticles. One type of Raman nanoparticle is coated with an antibody to bind to a biomarker of interest. The other type of Raman nanoparticle will contain no coating or a scrambled (i.e. nonspecific) coating. By analyzing the calibrated ratio of the signals from each type of Raman particle, we demonstrate accurate quantification of biomarker expression and effective suppression of nonspecific signals and background (See FIGS. 16A and 16B). The ratio metric image is obtained by applying the following equation to every pixel, C_{specific}-C_{nonspecific}. The concentration ratio between the targeted Raman nanoparticle (specific) versus the nonspecific control Raman nanoparticle is given by the following equation when applied to each pixel, C_{specific}/C_{nonspecific}. In the absence of specific binding and C_{specific}/C_{nonspecific}1. Therefore displaying the ratiometric image allows for more effective image interpretation in which zero specific binding is indicated as zero image intensity.

### Circumferential Scanning.

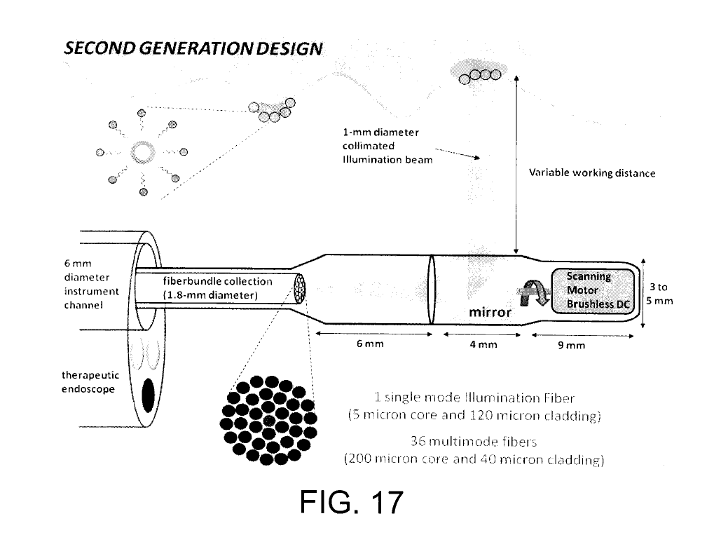

The addition of a 45-deg scanning mirror, actuated with a 1.9-mm diameter micromotor, will be used to perform circumferential scans of the lumen of the colon at a rate of 1 revolution/s (spatial resolution ˜1 mm) (See FIG. 17). The average colon has a radius between 2.5 and 3 cm, so having a working distance that is on the same order is important for the circumferential scanning device.

Schematic of another embodiment of the Raman endoscope.

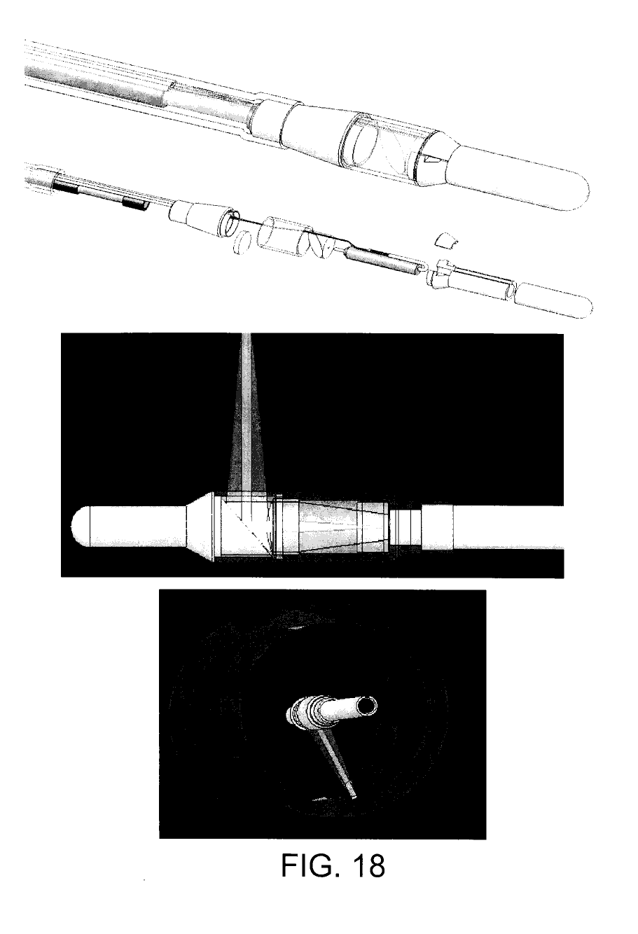

Notice how the Raman component would be inserted through the 6-mm accessory channel of a therapeutic colonoscope (FIG. 18). The endoscope can include a fiber-optic bundle with a single excitation fiber and a bundle of collection fibers for maximum signal collection. A scanning motor would allow for circumferential imaging of the colon wall. Axial pull-back of the endoscope will allow for imaging of the entire lumen of the colon.

### Additional Embodiments.

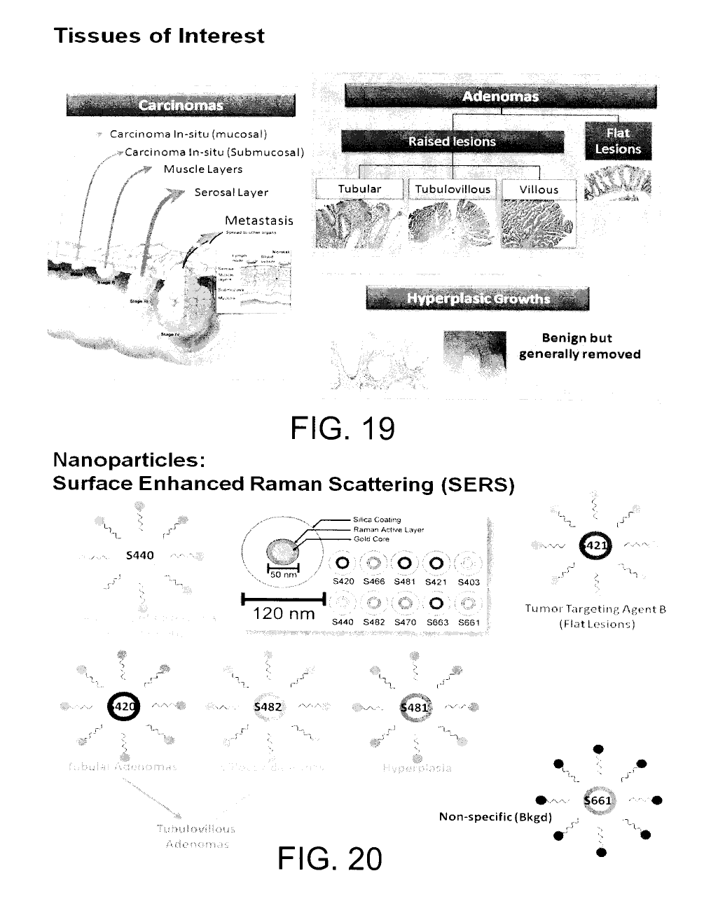

In an embodiment, the device can be used to distinguish a variety of tissues of interest in the colon such as: tubular adenomas, villous adenomas, tubulovillis adenomas, flat lesions, mucosal carcinoma in-situ, submucosal carcinoma in-situ, and more advanced carcinomas (See FIG. 19 and FIG. 20). In an embodiment, one type of Raman active agent may be targeted at tubular adenomas, and another at villous adenomas. If both types of agents are present (by a given ratio) you can conclude that in that region tubulovillis adenoma is present. In an embodiment, a single Raman active agent can be coated with multiple targeting agents. For instance, one type of Raman active agent can be coated with targeting agents targeting both all types of adenomas and all types of carcinomas. Or, one type of Raman active agent can be coated with a targeting agent for flat lesions and mucosal carcinoma in-situ only.

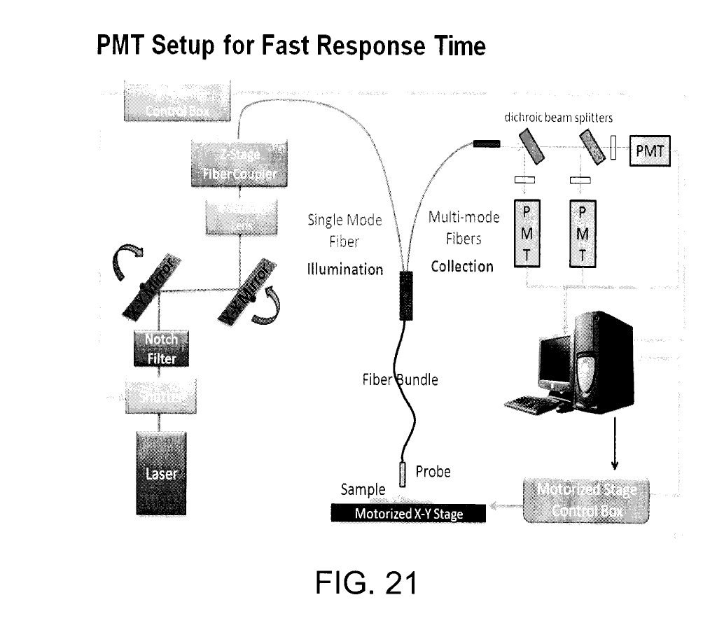

In an embodiment, a PMT and dichroic mirrors can be used instead of a spectrometer and CCD to allow for faster response time and cheaper components (See FIG. 21).

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5% ” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. In an embodiment, the term “about” can include traditional rounding based on numerical value and the measurement techniques. In addition, the phrase “about ‘x’ to ‘y’” includes “about ‘x’ to about ‘y’”.

It should be emphasized that the above-described embodiments of the present disclosure are merely possible examples of implementations, and are set forth only for a clear understanding of the principles of the disclosure. Many variations and modifications may be made to the above-described embodiments of the disclosure without departing substantially from the spirit and principles of the disclosure. All such modifications and variations are intended to be included herein within the scope of this disclosure.

We claim:

1. A method of imaging, comprising:
administering at least a first type of Raman agent to a subject, wherein the Raman agent has an affinity for a specific target;
introducing a Raman imaging device to an area of the subject;
exposing the area to a light beam from the Raman imaging device, wherein the light beam is scattered by the first type of Raman agent that is associated with the specific target, wherein the light beam that is scattered is referred to as a Raman scattered light energy;
detecting the Raman scattered light using the Raman imaging device; and
using the Raman scattered light energy to form an image.

2. The method of claim 1, further comprising mapping the area by circumferential scanning combined with a controlled retraction.

3. The method of claim 1, further comprising oscillating a mirror back and forth through a given angle to allow for a large area to be scanned.

4. The method of claim 1, further comprising positioning the mirror onto a specific area to image an area during a biopsy.

5. The method of claim 1, further comprising administering the first type of Raman agent to a subject and a second type of Raman agent to the subject.

6. The method of claim 5, wherein the first type of Raman agent has an affinity for tubular adenomas and the second type of Raman agent has an affinity for villous adenomas, wherein if both types of agents are present in a certain ratio, then tubulovillis adenoma is present at the area.

7. The method of claim 5, wherein the second agent does not have a specific affinity, and further comprising detecting the Raman scattered light from both the first agent and the second agent using the Raman imaging device, using the Raman scattered light energy from both the first and second Raman to form an image, wherein the second agent is used to normalize a Raman scattered light background signal.

8. The method of claim 1, wherein administering includes disposing the first Raman agent on the surface of the area.

9. The method of claim 8, wherein the area is washed to remove unattached Raman agents.

10. The method of claim 1, wherein the Raman agent is selected from the group consisting of: Surface Enhanced Raman Scattering (SERS) nanoparticle, composite organic inorganic nanoparticles (COINS), Single walled nanotubes (SWNTs), and a combination thereof.

11. The method of claim 1, wherein the Raman agent is a Surface Enhanced Raman Scattering (SERS) nanoparticle.

12. A method of performing Raman imaging, comprising:
providing, simultaneously, an untargeted Raman agent and a targeted Raman agent to a subject; and evaluating the ratio of Raman scattered light signals from the targeted and the untargeted Raman agents in an area, wherein the ratio provides an estimated measurement of truly bound Raman agents, wherein the measurement is substantially independent of the free-space optical working distance to the sample.

13. A method of imaging, comprising:
introducing a Raman imaging device to the subject;
positioning the Raman imaging device adjacent the specific target;
exposing the area to a light beam from the Raman imaging device, wherein the light beam is scattered by the tissue in the area, wherein the light beam that is scattered is referred to as Raman scattered light energy; and
detecting the Raman scattered light using the Raman imaging device, wherein the Raman scattered light energy is used to form an image.

14. A Raman imaging system for inspection of a sample comprising:
a light source;
a Raman detection system;
an optical fiber system to guide light derived from the light source to the sample and to further guide Raman scattered light energy from the sample to the Raman detection system; and
an optic system between the optical fiber system and the sample to concentrate said light onto the sample and to further collect the Raman scattered light energy from the sample, wherein the optic further concentrates the collected Raman scattered light energy onto the optical fiber system.

15. The Raman imaging system of claim 14, wherein:
the optical fiber system includes:
a first optical fiber system to guide light from the light source to the sample, and a second optical fiber system to guide Raman scattered light energy from the sample to the Raman detection system; and
wherein the optic system includes:
a first optic to concentrate the light onto the sample along a first axis; and
a second optic to collect the Raman scattered light energy from the sample along a second axis.

16. (canceled)

17. (canceled)

18. (canceled)

19. The Raman imaging system of claim 15, wherein the first axis and the second axis are non-collinear.

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. The Raman imaging system of claim 15, wherein the light concentrated by the first optic forms an illumination spot on the sample.

36. The Raman imaging system of claim 35, wherein the illumination spot is substantially centered about the first axis and the second axis.

37. The Raman imaging system of claim 36, wherein the first axis and the second axis are non-collinear.

38. The Raman imaging system of claim 35, wherein the illumination spot remains stationary with respect to the sample.

39. The Raman imaging system of claim 35, wherein the illumination spot is moved along a path with respect to the sample by a scanning means, whereby the path forms an illumination pattern on the sample.

40. The Raman imaging system of claim 39, wherein the illumination pattern is selected from the group consisting of: a linear pattern, a circular pattern, a partial circular pattern, an elliptical pattern, a helical pattern, and a raster pattern.

41. (canceled)

42. A Raman imaging system for inspection of a sample, comprising:
a light source;
a Raman detection system;
a first optical fiber system to guide light from the light source to the sample;
a second optical fiber system to guide Raman scattered light energy from the sample to the Raman detection system, wherein, the second optical fiber system has a proximal end adjacent to said Raman detection system; and
a first optic to concentrate said light onto the sample along a first axis.

43. (canceled)

44. (canceled)
